(19) United States
(12) Patent Application Publication
(10) Pub. No.: US 2021/0260341 A1
(43) Pub. Date: Aug. 26, 2021

(54) METHOD FOR COLLECTING TISSUE IN BILE DUCT

(71) Applicant: OLYMPUS CORPORATION, Tokyo (JP)

(72) Inventor: Mariko KANEKO, Tokyo (JP)

(73) Assignee: OLYMPUS CORPORATION, Tokyo (JP)

(21) Appl. No.: 16/795,793

(22) Filed: Feb. 20, 2020

(51) Int. Cl. A61M 25/01; A61M 25/06; A61M 25/00

(52) U.S. Cl. CPC A61M 25/0155; A61M 25/0662; A61M 2210/1042; A61M 25/0108; A61M 2025/0681; A61M 25/0082

(57) ABSTRACT

In a method of collecting a tissue in a bile duct, the method includes a step A of inflating a balloon in the bile duct and pressing the balloon against a wall of the bile duct, a step B of, in a state where a guide tube is positioned by using the balloon, bending a distal end portion of the guide tube so that a distal end opening of the guide tube is directed toward the wall of the bile duct, a step C of inserting a treatment tool into the guide catheter and protruding a distal end portion of the treatment tool toward the wall, and a step D of collecting a tissue using the treatment tool.

### BACKGROUND OF THE INVENTION

### Field of the Invention

The present invention relates to a method of collecting a tissue in a bile duct.

### Description of Related Art

Various examinations such as biopsy are performed on a tissue in a bile duct collected trans-endoscopically. When the tissue is collected in the bile duct trans-endoscopically, forceps and the like are protruded from an endoscope introduced near a duodenal papilla, and the forceps are inserted into the bile duct to collect the tissue.

Since the forceps inserted into the bile duct are in a state of being substantially parallel to a wall of the bile duct, the tissue cannot be sufficiently collected simply by opening and closing the forceps.

Japanese Unexamined Patent Application, First Publication No. 2005-279072 discloses forceps having a balloon attached to a distal end portion. When the balloon is inflated within a tubular organ, the forceps approach a tube wall on a side opposite to the balloon, and a tissue is easily collected by the forceps.

### SUMMARY OF THE INVENTION

According to an aspect of the present invention, there is provided a method of collecting a tissue in a bile duct, the method including: a step A of inflating a balloon in the bile duct and pressing the balloon against a wall of the bile duct; a step B of, in a state where a guide tube is positioned by using the balloon, bending a distal end portion of the guide tube so that a distal end opening of the guide tube is directed toward the wall of the bile duct; a step C of inserting a treatment tool into the guide tube and protruding a distal end portion of the treatment tool toward the wall; and a step D of collecting a tissue using the treatment tool.

### BRIEF DESCRIPTION OF THE DRAWINGS

FIG. 1 is a view illustrating a state in which a balloon catheter is inserted into a bile duct.

FIG. 2 is a view illustrating a step A of a method of collecting a tissue in a bile duct according to the first embodiment of the present invention.

FIG. 3 is an enlarged cross-sectional view of a distal end portion of a guide tube according to the collecting method.

FIG. 4 is a view illustrating a step B of the collecting method.

FIG. 5 is a view illustrating a step C of the collecting method.

FIG. 6 is a view illustrating the step B using another guide tube.

FIG. 7 is a view schematically illustrating a treatment tool according to a second embodiment of the present invention.

FIG. 8 is a view illustrating one process of a method of collecting a tissue in a bile duct using the treatment tool.

FIG. 9 is a view illustrating one process of the collecting method.

FIG. 10 is a view illustrating another example of a balloon.

FIG. 11 is a view illustrating another example of a balloon.

FIG. 12 is a view illustrating another example of a balloon.

FIG. 13 is a view illustrating another example of a balloon.

FIG. 14 is a view illustrating one process of a method of collecting a tissue in a bile duct according to the second embodiment of the present invention.

FIG. 15 is a view illustrating a step A1 of the collecting method.

FIG. 16 is a view illustrating a step B1 of the collecting method.

FIG. 17 is a view illustrating one process of the collecting method.

### DETAILED DESCRIPTION OF THE INVENTION

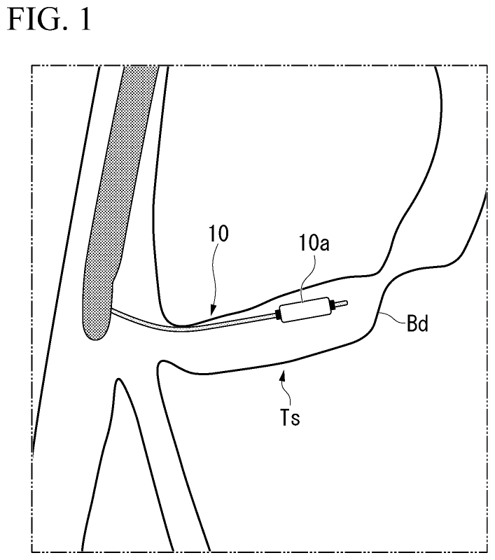

A first embodiment of the present invention will be described with reference to FIGS. 1 to 6.

In a method of collecting a tissue in a bile duct according to the present embodiment, distal end portions of a biopsy forceps are directed in a direction of a bile duct wall by combining a balloon and a guide tube.

As a preparation work, an endoscope is inserted through a mouth and a nose of a subject, and a distal end portion of the endoscope is moved to the vicinity of a duodenal papilla. Preferably, the endoscope is a side-view type endoscope having an elevator, but may be of another type.

Next, a contrast tube is inserted into the bile duct, and a contrast agent is injected into the bile duct via the contrast tube. After the contrast agent is injected, the contrast tube is removed from a body. A surgeon specifies a target site Ts from which a tissue is to be collected while observing an X-ray fluoroscopic image or the like in advance.

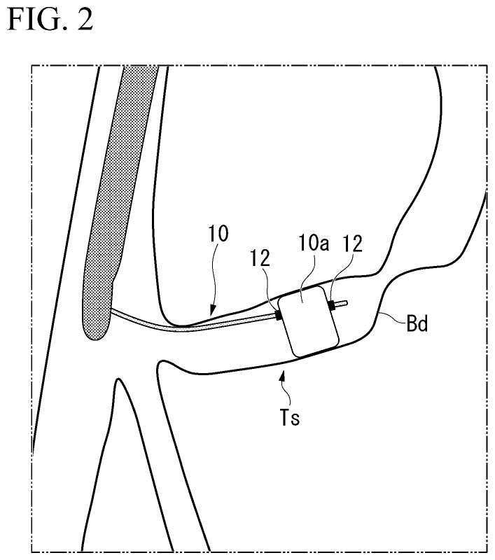

Next, a balloon catheter is inserted into a channel of the endoscope. As illustrated in FIG. 1, the balloon catheter 10 protruding from the distal end portion of the endoscope is inserted into a bile duct Bd. The injection of the contrast agent into the bile duct does not necessarily need to be performed via the contrast tube, but may be performed via the balloon catheter, for example. In this case, the contrast agent is injected into the bile duct in a state where a distal end of the balloon catheter is inserted into the bile duct. While the surgeon observes the X-ray fluoroscopic image or the like, the surgeon confirms a positional relationship between the target site Ts and a balloon 10a, and positions the balloon 10a closer to a liver (backward) than the target site Ts. Then, a fluid is supplied to the balloon 10a to inflate the balloon as illustrated in FIG. 2 (step A). The inflated balloon 10a is pressed against a wall of the bile duct. The fluid supplied to the balloon 10a includes the contrast agent. For example, the fluid is a liquid in which a contrast agent and physiological saline are mixed at a ratio of 1:1. Since concentrations of contrast materials are different from each other between the contrast agent injected into the bile duct Bd and the contrast agent in the balloon 10a, the surgeon can easily confirm a balloon shape including an outer shape of a proximal end portion of the balloon in an X-ray fluoroscopic image or like. Since ring-shaped X-ray opaque markers 12 are attached to both ends (preferably, on a shaft near the balloon 10a) of the balloon 10a, positions of both ends of the balloon 10a can be easily confirmed under X-ray fluoroscopy. Preferably, a material of the marker 12 is tantalum, tungsten, platinum, gold, or the like.

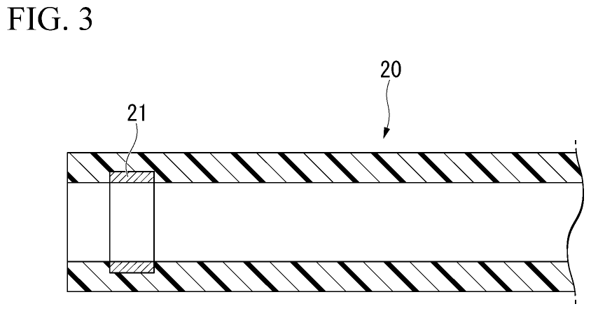

Next, a guide catheter (guide tube) is protruded from the endoscope and inserted into the bile duct. FIG. 3 illustrates an enlarged cross-sectional view of a distal end portion of a guide tube 20. A ring-shaped X-ray opaque tip 21 is disposed at the distal end portion of the guide tube 20. As a material of the X-ray opaque tip 21, tantalum, tungsten, platinum, gold, or the like is preferable. Instead of the X-ray opaque tip 21, an X-ray opaque marking may be provided on an outer peripheral surface of the guide tube 20. Preferably, the marking is formed by applying an ink containing at least one metal selected from a group consisting of tantalum, tungsten, platinum, and gold.

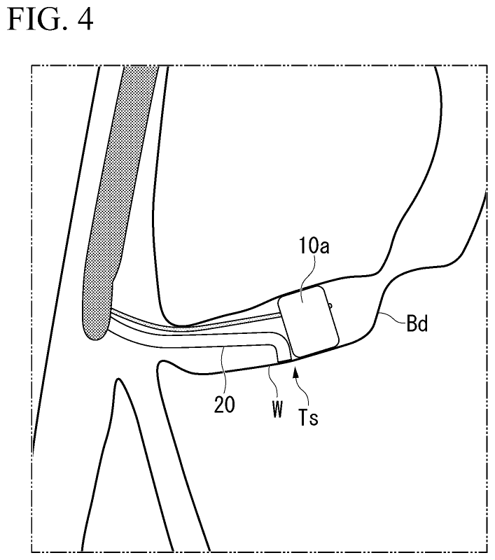

The surgeon brings a distal end of the guide tube 20 into contact with the inflated balloon 10a. Thereafter, the surgeon pushes the guide tube 20. When the surgeon pushes the guide tube 20, the distal end portion of the guide tube 20 is bent while sliding on the outer surface of the balloon 10a. As a result, as illustrated in FIG. 4, the distal end portion of the guide tube 20 is bent toward a wall W of the bile duct. In this state, a distal end opening of the guide tube 20 is directed toward the wall W of the bile duct. The surgeon secures a gap between the distal end opening of the guide tube 20 and the bile duct wall while maintaining a state where the distal end opening is directed toward the wall of the bile duct while the guide tube 20 is held (step B).

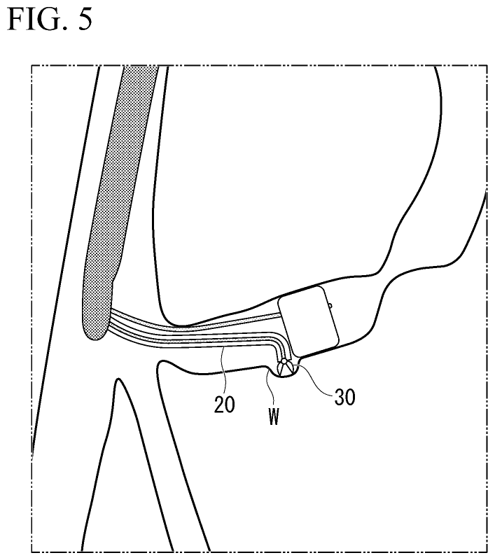

Next, the surgeon inserts the biopsy forceps from a proximal end of the guide tube 20 and moves the biopsy forceps forward. Then, as illustrated in FIG. 5, a distal end portion of biopsy forceps 30 protrudes from the distal end opening of the guide tube 20 (step C). In the step B, since the distal end opening of the guide tube 20 is directed toward the wall W of the bile duct, the distal end portion of the biopsy forceps 30 protrudes toward the wall W.

The surgeon brings the distal end portion of the biopsy forceps 30 into contact with the wall W of the bile duct, and operates the biopsy forceps 30 to collect the tissue of the bile duct (step D). Since the distal end portion of the biopsy forceps 30 protrudes toward the wall W of the bile duct, the distal end portion of the biopsy forceps 30 can come into contact with the wall W of the bile duct only by moving the biopsy forceps 30 forward.

After the step D, the surgeon removes the biopsy forceps 30 and collects the tissue. In a case where an amount of tissue is not sufficient, or in a case where the tissue at another site is to be collected, the biopsy forceps 30 is inserted into the guide tube 20 again, and the step D is performed. If necessary, the guide tube 20 may be rotated around a longitudinal axis, and thus, the balloon 10a and the guide tube 20 may be moved forward or rearward.

As described above, according to the method of collecting the tissue in the bile duct according to the present embodiment, in the step B, the distal end opening of the guide tube 20 from which the biopsy forceps 30 protrude is directed toward the wall of the bile duct. Accordingly, by only inserting the forceps 30 into the guide tube 20 and moving the forceps 30 forward, the biopsy forceps 30 can be protruded toward the wall W of the bile duct. As a result, the distal end portion of the biopsy forceps 30 can approach the wall W of the bile duct in a direction nearly perpendicular to the wall W to collect the tissue.

In general, in most cases, the guide catheter is formed of a resin, and the biopsy forceps are formed of a metal. Accordingly, in most cases, biopsy forceps are more rigid than the guide catheter. In this case, even if the distal end opening of the guide catheter is bent toward the wall of the bile duct, when the forceps enter the guide catheter, a rigidity of the guide catheter loses due to a rigidity of the forceps and the guide catheter is straightened, and there is a possibility that a direction of the distal end opening is changed.

In the collecting method of the present embodiment, in the step A, the balloon 10a is inflated on a back side of the guide tube 20, and a position of the distal end portion of the guide tube 20 during a procedure is positioned. Thereafter, in the step B, the distal end portion of the guide tube 20 comes into contact with the balloon 11. As a result, even when a force for strengthening the guide tube 20 acts due to the rigidity of the biopsy forceps 30, the balloon 10a receives the force and the direction of the distal end opening of the guide tube 20 is held without being changed. Therefore, even when the biopsy forceps 30 having a rigidity higher than that of the guide tube 20 are used, the biopsy forceps 30 can protrude toward the wall W of the bile duct.

According to the technique described in Japanese Unexamined Patent Application, First Publication No. 2005-279072, the direction of the distal end portion of the forceps can be changed by inflating the balloon in a relatively large tubular organ such as a large intestine. However, in a thin tubular organ such as a bile duct, since there is not enough space around the forceps, even when the balloon provided on the forceps is inflated, the forceps approach the wall of the tubular organ remain in a state where the forceps are substantially parallel to traveling of a tubular organ. Accordingly, the distal end portion of the forceps is not directed toward the wall. Therefore, the technique described in Japanese Unexamined Patent Application, First Publication No. 2005-279072 is not effective when the forceps are used in a narrow luminal organ.

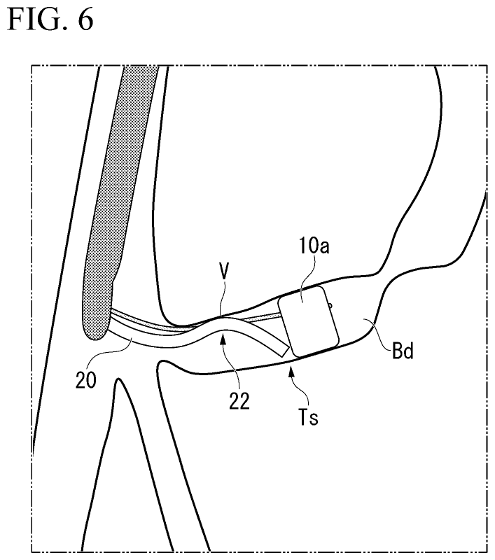

In the present embodiment, a distal end side of the guide tube 20 may have a pre-curve 22 that is bent in advance. Since a diameter of the bile duct Bd is about several millimeters, when the distal end of the guide tube 20 having the pre-curve 22 is pressed against the balloon 10a, as illustrated in FIG. 6, the guide tube 20 is deformed into an S shape and comes into contact with both a facing wall V of the target site Ts and an outer peripheral surface of the balloon 10a. As a result, the position of the guide tube 20 is easily stabilized. By positioning the guide tube 20, the distal end opening of the guide tube 20 can be brought closer to a front surface of the target site Ts, and the tissue collection by the biopsy forceps 30 becomes easier.

If the balloon catheter and the guide tube can be relatively moved in a longitudinal direction, the step B can be performed. Accordingly, the method of collecting the tissue can be performed. The balloon catheter and the guide tube may be passed through the same channel of the endoscope, or may be passed through different channels in a case where the endoscope has a plurality of channels. Furthermore, one of the balloon catheter and the guide tube may be held outside the endoscope by a plurality of rings or the like along an insertion portion of the endoscope.

The guide tube may be inserted into the bile duct before the balloon catheter, or the guide tube and the balloon catheter may be inserted into the bile duct at the same time.

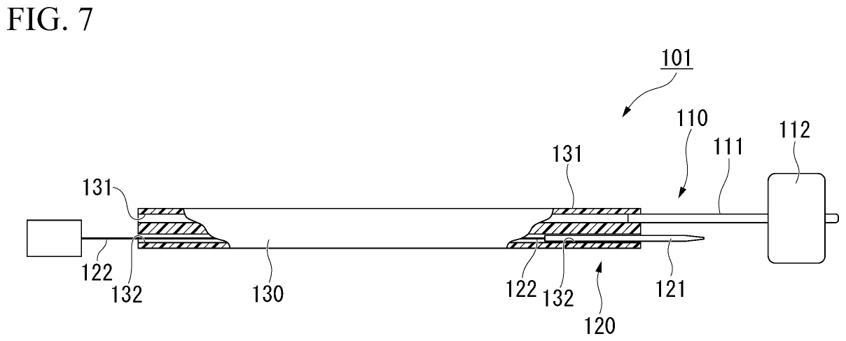

A second embodiment of the present invention will be described with reference to FIGS. 7 to 9. In this embodiment, a treatment tool suitable for collecting the tissue in the bile duct in combination with an endoscope having only one channel is illustrated.

FIG. 7 illustrates a treatment tool 101 according to the present embodiment. The treatment tool 101 has a configuration in which a balloon catheter 110 and a guide tube 120 are integrated with each other.

The treatment tool 101 includes a double lumen tube 130. The double lumen tube 130 has two lumens which are a first lumen 131 and a second lumen 132. The first lumen 131 and the second lumen 132 extend in a longitudinal direction of the double lumen tube 130.

An extension tube 111 is airtightly connected and fixed to a distal end opening of the first lumen 131. A balloon 112 is connected to the extension tube 111. Therefore, when a fluid flows into the first lumen 131, the fluid can be supplied to the balloon 112 via the extension tube 111 to inflate the balloon. That is, in the treatment tool 101, the balloon 112, the extension tube 111, and the first lumen 131 constitute the balloon catheter 110.

The guide tube 120 includes a tubular tip catheter 121 and a wire 122 which is connected to the tip catheter 121. The tip catheter 121 is inserted into an opening of the second lumen 132 and can be protruded from a distal end opening of the second lumen 132. The wire 122 passes through the second lumen 132 and protrudes from a proximal end opening of the second lumen 132. Therefore, when the wire 122 is moved forward or rearward in a longitudinal direction, the tip catheter 121 can be relatively moved with respect to the double lumen tube 130 and the balloon 112.

Since a length of the tip catheter 121 is longer than that of the extension tube 111 located outside the double lumen tube 130, even in a state where a distal end of the tip catheter 121 is in contact with the balloon 112, a proximal end portion of the tip catheter 121 is located in the second lumen. 132.

A flow of collecting the tissue of the present embodiment using the treatment tool 101 will be described.

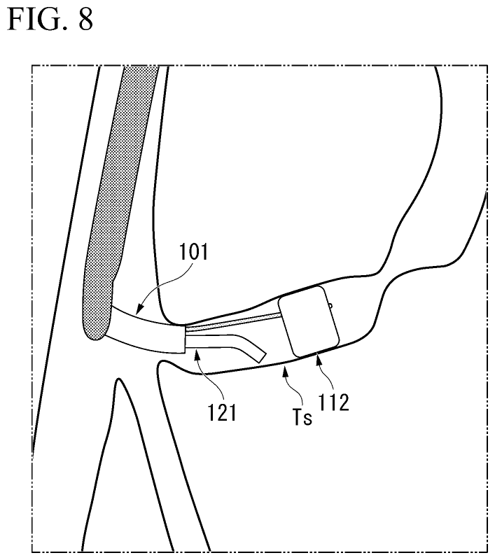

First, in a state where the balloon 112 is deflated, the treatment tool 101 is inserted into a channel of an endoscope. A distal end side of the treatment tool 101 protrudes from the channel of the endoscope, and the balloon 112 is inserted into the bile duct. The target site Ts is determined in the same procedure as in the first embodiment, the balloon 112 is located at a position deeper than that of the target site Ts, and the balloon 112 is inflated as illustrated in FIG. 8 (step A).

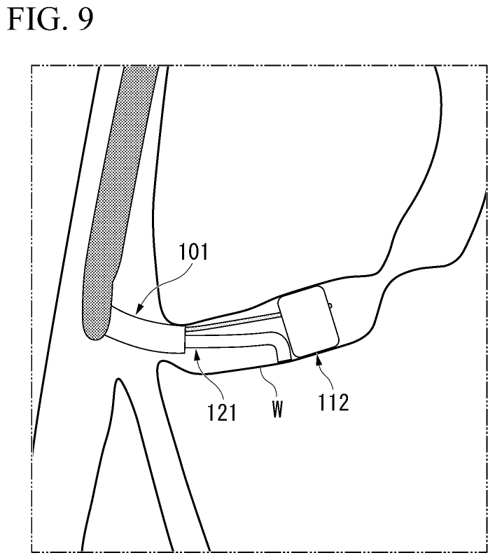

In the step B, the surgeon operates the wire 122 to move the tip catheter 121 forward so that the tip catheter 121 comes into contact with the inflated balloon 112. Then, as illustrated in FIG. 9, similarly to the first embodiment, the distal end of the tip catheter 121 is bent so that the distal end opening is directed toward the wall W of the bile duct. The surgeon holds the wire 122 to maintain an orientation of the distal end opening.

In the step C, the surgeon inserts the biopsy forceps 30 into the second lumen 132 and moves the biopsy forceps forward. The biopsy forceps 30 enters the tip catheter 121 within the second lumen 132 and protrudes from the distal end opening of the tip catheter 121. Since the distal end portion of the biopsy forceps 30 is directed toward the wall W, the step D can be performed as in the first embodiment. That is, in the treatment tool 101, the tip catheter 121 which is a portion of the guide tube 120 is configured to be relatively movable with respect to the balloon 112.

In the method of collecting the tissue in the bile duct according to the present embodiment, as in the first embodiment, the distal end portion of the biopsy forceps 30 can come into easy contact with the wall W of the bile duct so that the tissue can be easily collected.

The above-described treatment tool 101 has the configuration in which the balloon 112 does not move and the tip catheter 121 which is a portion of the guide tube 120 can move. However, this relationship may be reversed. That is, a configuration may be adopted, in which the proximal end portion of the tip catheter 121 is fixed in a state where the proximal end portion is inserted into the distal end portion of the second lumen 132 of the double lumen tube 130, and the tube for supplying fluid to the balloon 112 is inserted into the first lumen 131, and the balloon 112 is relatively movable with respect to the double lumen tube 130 and the tip catheter 121.

In the treatment tool 101, the tube does not need to pass through the first lumen 131. Accordingly, it is possible to reduce a diameter of the first lumen 131. Further, the tip catheter 121 is inserted only into the distal end side of the double lumen tube 130. Accordingly, a diameter of the proximal end side of the second lumen 132 can be easily reduced. Therefore, a configuration of the treatment tool 101 is suitable for a case where an outer diameter of the double lumen tube 130 is to be reduced.

In the first and second embodiments, a shape of the balloon can be appropriately set. For example, a slope may be provided on a proximal end side of the balloon. When a straight guide tube moves forward, the distal end thereof may be sunk into the balloon. If the balloon has the shape having a slope on the proximal end side, the distal end of the guide tube is moved forward along the slope. Accordingly, even when the straight guide tube is provided, the direction of the distal end opening can be smoothly changed.

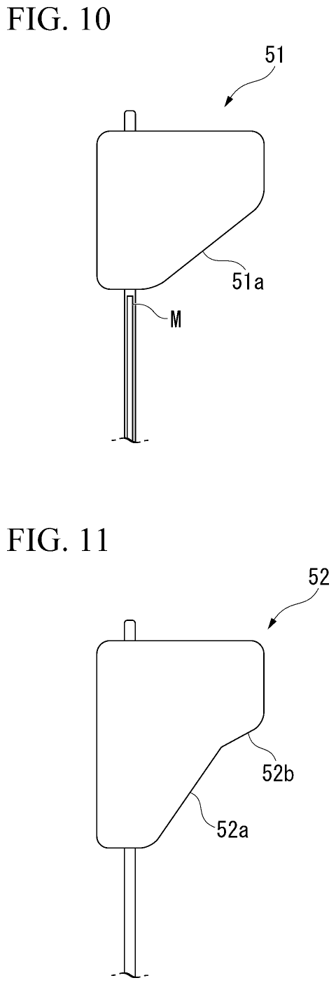

FIGS. 10 to 12 illustrate examples of a balloon having a slope on the proximal end side. A balloon 51 illustrated in FIG. 10 has a shape at the time of inflation in which a columnar end portion is cut off obliquely and has a slope 51a.

In FIG. 10, a marking M associated with the direction in which the slope 51a extends is formed only on a portion of the shaft in the circumferential direction of a shaft for moving the balloon 51 forward and rearward. The marking M extends along a longitudinal axis of the shaft, and a length of the marking M is set such that even when the balloon 51 is introduced into the bile duct, a part thereof is surely located in a duodenum.

The surgeon can identify the direction of the slope 51a by visually recognizing a direction of the marking M on the shaft protruding into the duodenum from the duodenal papilla with a duodenal scope. After the balloon 51 is inserted into the bile duct, the balloon 51 is disposed so that the orientation of the target site Ts and the slope 51a coincide with each other while the balloon 51 is checked with an X-ray fluoroscopic image or the like. As illustrated in FIG. 10, the proximal end portion of the balloon 51 is inclined toward the wall of the bile duct Bd in the inflated state of the balloon 51. Accordingly, when the surgeon pushes the guide tube, the distal end portion of the guide tube is easily bent while sliding on the outer surface of the balloon 51.

Means for specifying the direction of the slope 51a is not limited to the above-described marking M. For example, an X-ray opaque marking associated with the direction in which the slope 51a extends may be provided on a shaft near the slope 51a of the balloon 51. In this case as well, by checking the positional relationship between the marking position and the target site Ts in the X-ray fluoroscopic image, the balloon 51 can be disposed so that the target portion Ts and the direction of the slope 51a coincide with each other.

A slope of a balloon 52 illustrated in FIG. 11 has a first slope 52a and a second slope 52b connected to the first slope, and the slope is gradually increased. Accordingly, the direction of the distal end opening of the guide tube can be more smoothly changed. When the first slope 52a and the second slope 52b are arranged around the axis, an appropriate slope can be selected according to a bile duct inner diameter by rotating the balloon, and the step B can be appropriately performed according to the bile duct inner diameter.

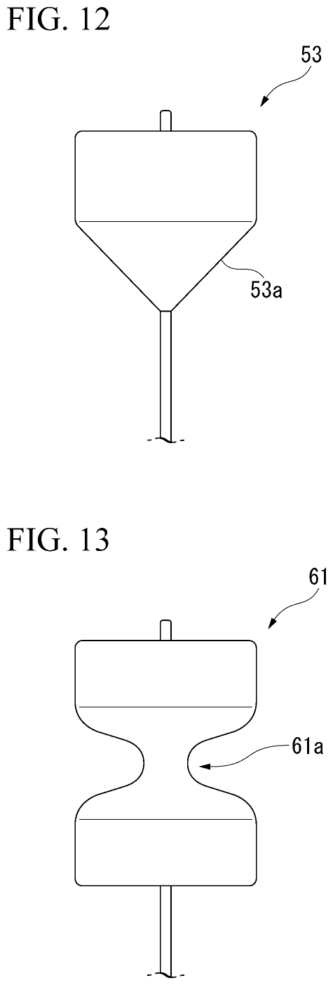

A balloon 53 illustrated in FIG. 12 has a conical proximal end and has a curved slope 53a. That is, the slope extends in all directions around a longitudinal axis with respect to the guide tube approaching the balloon. Accordingly, it is not necessary to rotate the balloon according to the direction in which the distal end opening is directed.

The slopes illustrated in FIGS. 10 to 12 may be provided in an appropriate combination.

The balloon may have a shape like a dumbbell in which an intermediate portion 61a in an axial direction is relatively thin like a balloon 61 illustrated in FIG. 13.

When the balloon 61 is inflated in the bile duct, the bile duct wall entering around the intermediate portion 61a. Accordingly, a contact area between the bile duct wall and the balloon 61 increases, and the position of the balloon 61 is stabilized. Therefore, it is difficult for the balloon 61 to move in the longitudinal direction of the bile duct after the balloon 61 is inflated.

The shape illustrated in FIG. 13 may be combined with the slopes illustrated in FIGS. 10 to 12.

A third embodiment of the present invention will be described. The guide tube in the third embodiment is a cholangioscope. A cholangioscope is an endoscope of which an outer diameter of an insertion portion is small enough to be inserted into a channel of an endoscope (duodenal scope) for observing the duodenum. The cholangioscope has a channel into which the biopsy forceps can be inserted and an observation optical system which can image a target site. The insertion portion has a bending portion which can be bent.

In the present embodiment, a balloon is attached to the insertion portion of the cholangioscope.

A method of collecting the tissue in a bile duct according to the present embodiment will be described.

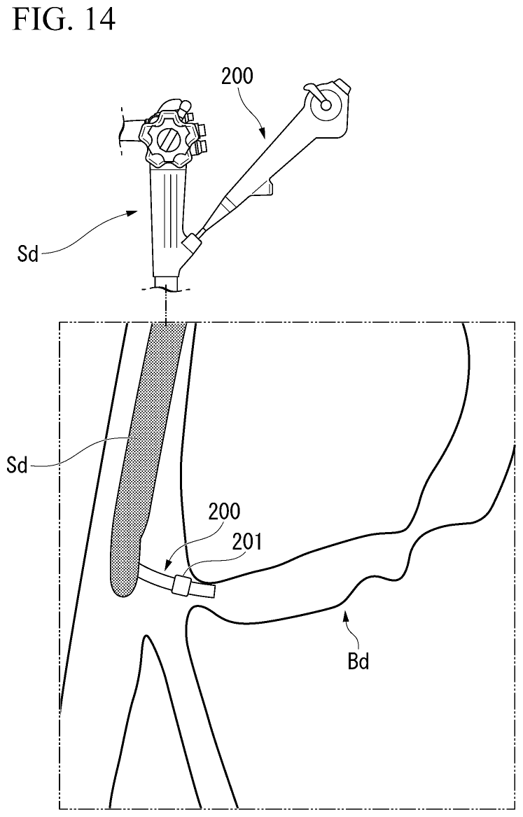

First, as illustrated in FIG. 14, the cholangioscope 200 is inserted into the bile duct Bd via a duodenal scope Sd (step 1). The above-described balloon 201 is fixed to a distal end portion of the cholangioscope 200 to be closer to a hand side than the bending portion 202.

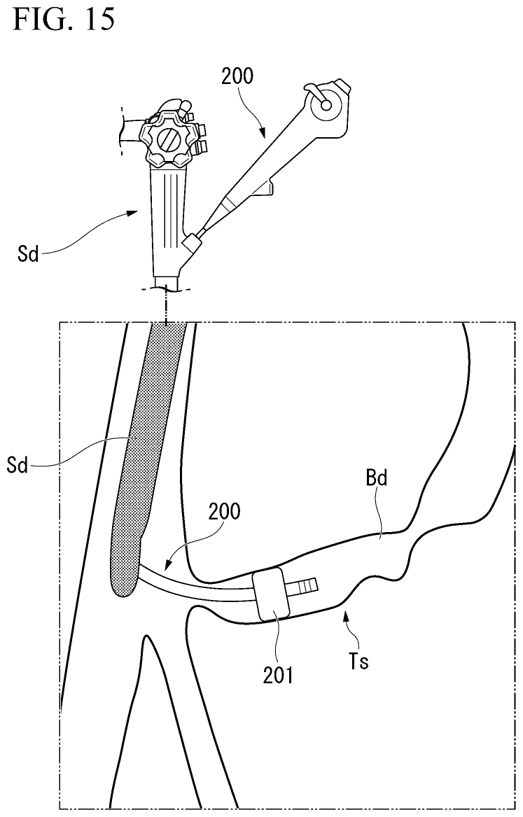

Next, as illustrated in FIG. 15, the balloon 201 is inflated in the bile duct Bd, and the balloon 201 is pressed against the bile duct wall. Thus, the cholangioscope 200 is positioned (step A1). The balloon 201 is inflated at a location where a distal end of the cholangioscope 200 is located closer to the hand side (the side of the duodenal papilla) than the target site Ts.

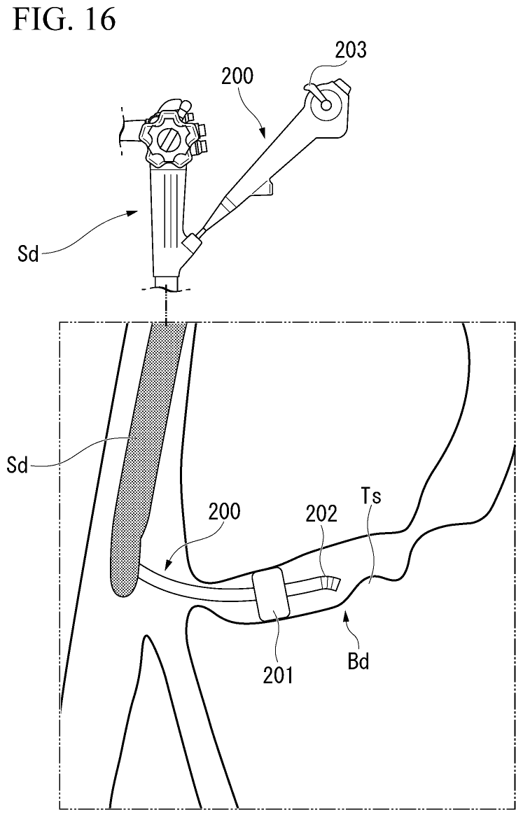

Next, as illustrated in FIG. 16, the bending portion 202 is bent by operating a knob 203 of the cholangioscope 200, and the distal end opening of the channel is directed toward the wall of the target site Ts (step B1).

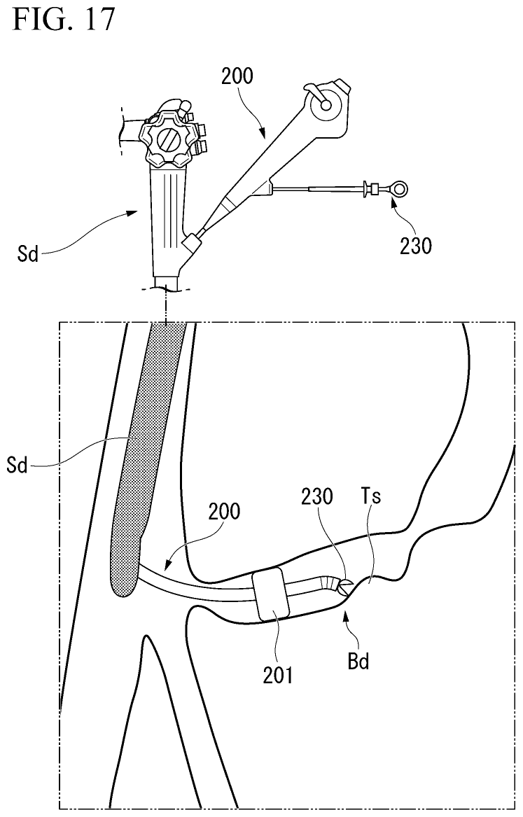

Next, as illustrated in FIG. 17, the tissue at the target site Ts is collected using the biopsy forceps 230 protruding from the distal end portion of the cholangioscope 200 (steps C1 and D1).

In the present embodiment, the above-described step B1 may be performed after the biopsy forceps 230 are inserted into the cholangioscope.

The cholangioscope may not include the bending portion or the balloon. In this case, the bile duct tissue can be collected by operating the cholangioscope in the same procedure as the guide catheter of the first embodiment.

As another example, a guide tube (a configuration in which the observation optical system is removed from the cholangioscope of the present embodiment) having the bending portion and the balloon may be used. In this case, as in the first embodiment, by performing the observation with an X-ray fluoroscopic image, the tissue in the bile duct can be collected in substantially the same procedure as in the third embodiment.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

The treatment tool inserted into the guide tube is not limited to the forceps. Other treatment tools such as a biopsy brush may be used as long as the tissue can be collected.

The invention claimed is:

1. A method of collecting a tissue in a bile duct, the method comprising:
inflating a balloon in the bile duct and pressing the balloon against a wall of the bile duct;
bending a first distal end portion of a guide tube so that a distal end opening of the guide tube is directed toward the wall of the bile duct to allow the guide tube to be positioned using the balloon;
inserting a treatment tool into the guide tube and protruding a second distal end portion of the treatment tool toward the wall; and
collecting a tissue using the treatment tool.

2. The method of claim 1, further comprising:
directing the distal end opening of the guide tube toward the wall of the bile duct while sliding the guide tube inserted into the bile duct on an outer surface of the balloon.

3. The method of claim 1,
wherein the first distal end portion of the guide tube is bent.

4. The method of claim 1,
wherein bending the first distal end portion of the guide tube includes moving the guide tube forward along an outer surface of the balloon.

5. The method of claim 1,
wherein the balloon has a slope on a proximal end side of the balloon.

6. The method of claim 1, further comprising:
collecting the tissue at a first position; and
inflating the balloon at a second position that is closer to a liver than the first position.

7. The method of claim 1, wherein: the guide tube includes:
the balloon, and
a bending portion which is provided on a distal end side from the balloon and is bendable, and
the method further comprises bending the bending portion so that the distal end opening of the guide tube is directed toward the wall of the bile duct.
